(19) United States
(12) Patent Application Publication
(10) Pub. No.: US 2025/0268717 A1
(43) Pub. Date: Aug. 28, 2025

(54) SYSTEM AND METHOD FOR TREATMENT AND PREVENTION OF PERIPROSTHETIC JOINT INFECTIONS

(71) Applicant: Osteal Therapeutics, Inc., Dallas, TX (US)

(72) Inventors: Brian de Beaubien, Dallas, TX (US); Jude Paganelli, Dallas, TX (US); Daniel Pflaster, Dallas, TX (US); Daivon Deans, Dallas, TX (US); Ryan Watson, Dallas, TX (US); Brian Bowman, Dallas, TX (US)

(21) Appl. No.: 19/043,036

(22) Filed: Jan. 31, 2025

Related U.S. Application Data
(62) Division of application No. 17/401,843, filed on Aug. 13, 2021, now Pat. No. 12,239,538.
(60) Provisional application No. 63/065,413, filed on Aug. 13, 2020.

(51) Int. Cl. A61F 2/30; A61F 2/36; A61F 2/38; A61F 2/40; A61M 1/00

(52) U.S. Cl. CPC A61F 2/30724; A61F 2/36; A61F 2/389; A61F 2/4014; A61M 1/90; A61F 2002/3006; A61F 2002/30672; A61F 2002/30677

(57) ABSTRACT

Provided herein are rapid and effective local infection therapy methods, systems, and devices that significantly reduce the mortality, morbidity, and the cost of care in rare musculoskeletal infections. Continuous delivery of antibiotic therapy locally, at the infection site, reduces edema and provides antibiotic irrigation, significantly improving outcomes while reducing the need for systemic antibiotics.

### CROSS-REFERENCE TO RELATED APPLICATIONS

This application is a divisional of U.S. patent application Ser. No. 17/401,843 filed Aug. 13, 2021, which claims the benefit of U.S. Provisional Patent Application No. 63/065,413 filed Aug. 13, 2020, the content of which are incorporated herein by reference.

### BACKGROUND

Each year, over one million Americans undergo hip or knee arthroplasty (joint replacement surgery) to treat reduced mobility and joint pain. Moreover, Total Should Replacement (TSR) volume is growing rapidly. Periprosthetic joint infections (PJI), a debilitating and hard to treat complication, affects ˜2% of this patient population. Individuals with hip or knee PJI have 6.5% mortality in the first year, a 3-fold increase over individuals the same age (2%).

Surgery with adjuvant systemic antibiotics is the foundation of PJI treatment surgical management of PJI however is complicated by the presence of a biofilm, a polysaccharide matrix generated by the bacteria, rendering them highly resistant to systemic antibacterial treatment. Metallic implants provide a site for biofilm formation and in most cases, implant removal is considered an essential part of surgical management to eradicate the infection. The intravenous (IV) administration of systemic antibiotics for PJI may not result in adequate therapeutic concentrations for eradication of biofilm at the site of infection. Achievement of locally therapeutic levels is crucial for clinical success; however, this is difficult or impossible due to the fact that most PJI pathogens are biofilm forming. Biofilm-encapsulated bacteria require minimum biofilm eradication concentrations (MBEC) of antibiotics that are several orders of magnitude (100 to 1000×) above the minimum inhibitory concentrations (MIC) sufficient to eradicate planktonic bacteria. Therapeutic target attainment at levels near the MBEC may not work via systemic routes of administration without significant risk of toxicity to other organ systems.

### SUMMARY

Provided herein is a hip spacer comprising a body comprising a fluid inlet; a fluid outlet; and a femoral head aperture. Also provided herein is a hip spacer system comprising: the hip spacer; and a catheter in fluidic communication with the inlet of the hip spacer. Also provided herein is a hip spacer platform comprising: the hip spacer system; and a pump in fluidic communication with the catheter and the fluid outlet of the hip spacer. Also provided herein is a hip spacing kit comprising: two or more sizes of the hip spacer system; and a pump in fluidic communication with the catheter and the fluid outlet of the hip spacer.

Provided herein is a knee spacer comprising a body comprising a fluid inlet and a fluid outlet. Also provided herein is a knee spacer system comprising: the knee spacer; and a catheter in fluidic communication with the inlet of the knee spacer. Also provided herein is a knee spacer platform comprising: the knee spacer system; and a pump in fluidic communication with the catheter and the fluid outlet of the knee spacer. Also provided herein is a knee spacing kit comprising: two or more sizes of the knee spacer system; and a pump in fluidic communication with the catheter and the fluid outlet of the knee spacer.

Provided herein is a shoulder spacer comprising a body comprising a fluid inlet and a fluid outlet. Also provided herein is a shoulder spacer system comprising: the shoulder spacer; and a catheter in fluidic communication with the inlet of the shoulder spacer. Also provided herein is a shoulder spacer platform comprising: the shoulder spacer system; and a pump in fluidic communication with the catheter and the fluid outlet of the shoulder spacer. Also provided herein is a shoulder spacing kit comprising: two or more sizes of the shoulder spacer system; and a pump in fluidic communication with the catheter and the fluid outlet of the shoulder spacer.

Also provided herein is a method of treating periprosthetic joint infection, the method comprising: providing one of: a hip spacer platform as disclosed herein; a knee spacer platform as disclosed herein; and a shoulder (or reverse shoulder) spacer platform disclosed herein; supplying a medication to the pump to irrigate a location of the joint infection over a period of time.

In another aspect, embodiments of the present invention encompass spacer platform systems and methods for treating a patient presenting with an acute periprosthetic joint infection of a joint. Exemplary spacer platforms can include a spacer system having a spacer and a catheter, where the spacer and catheter are configured for detachable coupling. A spacer platform can also include a pump assembly that is configured for coupling with the catheter. In some embodiments, the spacer is configured for placement between a first implant and a second implant of a permanent joint prosthesis, where the first implant secured with a first bone of the joint and the second implant secured with a second bone of the joint. In some embodiments, the spacer includes a first surface configured for articulating engagement with the first implant, a second surface configured for fixed engagement with the second implant, an exposed surface disposed between the first surface and the second surface, and an inlet configured to receive a treatment fluid. In some embodiments, the first surface includes a first plurality of outlets in fluid connection with the inlet and the exposed surface includes a second plurality of outlets in fluid communication with the inlet, such that treatment fluid delivered into the inlet can flow out through the first plurality of outlets and to the first implant and out through the second plurality of outlets and into a joint space between the first implant and the second implant.

In some instances, the spacer system is a hip spacer system, the joint is a hip joint, the first implant is an acetabular cup, and the second implant is a femoral stem. In some instances, the spacer system is a knee spacer system, the joint is a knee joint, the first implant is a femoral implant, and the second implant is a tibial implant. In some instances, the spacer system is a shoulder spacer system, the joint is a shoulder joint, the first implant is a glenoid implant, and the second implant is a humeral stem. In some instances, the second surface of the spacer includes a plurality of fluted channels. In some instances, at least one of the fluted channels includes a plurality of fluid outlets. In some instances, the plurality of fluted channels are arrayed about a center axis of the spacer. In some instances, the plurality of fluted channels enable fluid flow under the spacer, between the spacer and the second implant. In some instances, the spacer is undersized relative to the first implant. In some instances, the spacer comprises a biocompatible polymer such as low-density polyethylene (LDPE), high-density polyethylene (HDPE), polypropylene (PP), polyvinyl chloride (PVC) polystyrene (PS) nylon, polytetrafluoroethylene, a thermoplastic polyurethane (TPU), acetyl copolymer, silicone, polyether ether ketone (PEEK), a polyurethane, a biocompatible elastomer, or ultrahigh molecular weight polyethylene (UHMWPE).

In still another aspect, embodiments of the present invention encompass methods for treating a patient presenting with an acute periprosthetic joint infection of a joint. Exemplary methods can include removing one or more prosthetic components disposed between a first implant of secured with a first bone of the joint and a second implant secured with a second bone of the joint, placing a spacer of a spacer system between the first implant and the second implant, the spacer system having the spacer, a catheter, and a pump assembly, where the spacer and catheter are configured for detachable coupling, and where the pump assembly is configured for coupling with the catheter. Methods can also include delivering a treatment fluid from the pump, through the catheter, into an inlet of the spacer, and out through a first plurality of outlets at a first surface of the spacer to the first implant, and out through a second plurality of outlets at an exposed surface of the spacer and into a joint space between the first implant and the second implant. In some cases, the spacer system is a hip spacer system, the joint is a hip joint, the first implant is an acetabular cup, and the second implant is a femoral stem. In some cases, the spacer system is a knee spacer system, the joint is a knee joint, the first implant is a femoral implant, and the second implant is a tibial implant. In some cases, the spacer system is a shoulder spacer system, the joint is a shoulder joint, the first implant is a glenoid implant, and the second implant is a humeral stem. In some cases, the second surface of the spacer includes a plurality of fluted channels. In some cases, at least one of the fluted channels includes a plurality of fluid outlets. In some cases, the plurality of fluted channels are arrayed about a center axis of the spacer. In some cases, the spacer is undersized relative to the first implant. In some cases, the spacer includes a biocompatible polymer such as low-density polyethylene (LDPE), high-density polyethylene (HDPE), polypropylene (PP), polyvinyl chloride (PVC) polystyrene (PS) nylon, polytetrafluoroethylene, a thermoplastic polyurethane (TPU), acetyl copolymer, silicone, polyether ether ketone (PEEK), a polyurethane, a biocompatible elastomer, or an ultrahigh molecular weight polyethylene (UHMWPE). In some cases, the pump assembly is a negative pressure wound therapy pump assembly.

In yet another aspect, embodiments of the present invention encompass methods of treating a patient presenting with an acute periprosthetic joint infection of a joint, where methods may include removing one or more prosthetic components disposed between a first implant of secured with a first bone of the joint and a second implant secured with a second bone of the joint, and placing a spacer of a spacer system between the first implant and the second implant. The spacer system can include the spacer, a catheter, and a pump assembly, and the spacer and catheter can be configured for detachable coupling. The pump assembly can be configured for coupling with the catheter. Methods may also include delivering an antibiotic treatment fluid from the pump, through the catheter, into an inlet of the spacer, and out through a first plurality of outlets at a first surface of the spacer to the first implant, out through a second plurality of outlets at an exposed surface of the spacer and into a joint space between the first implant and the second implant, and out through a third plurality of outlets at a second surface of the spacer, the third plurality of outlets disposed within one or more fluted channels of the second surface so as to provide fluid flow between the spacer and the second implant. In some cases, the antibiotic treatment fluid is provided continuously or periodically to the patient over a treatment period of 7 days or more. In some cases, the catheter remains attached with the spacer throughout the treatment period.

In still yet another aspect, embodiments of the present invention encompass spacer platforms for treating a patient presenting with an acute periprosthetic joint infection of a joint, where spacer platforms can include a spacer system having a spacer and a catheter, and where the spacer and catheter are configured for detachable coupling. In some cases, a pump assembly that is configured for coupling with the catheter to deliver a treatment fluid to the joint. In some cases, the spacer includes a first surface configured for articulating engagement with the first implant, a second surface configured for fixed engagement with the second implant, an exposed surface disposed between the first surface and the second surface, and an inlet configured to receive the treatment fluid. In some cases, the spacer is configured with a plurality of outlets to deliver the treatment fluid into the joint.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a front-view illustration of an exemplary hip antibiotic irrigation platform, per an embodiment herein;

FIG. 2 shows a front-view illustration of an exemplary hip antibiotic irrigation system installed about a human hip joint, per an embodiment herein;

FIG. 3A shows a front-view illustration of an exemplary hip spacer system, per an embodiment herein;

FIG. 3B shows a cross-sectioned front-view illustration of an exemplary hip spacer system, per an embodiment herein;

FIG. 4A shows a top-view illustration of an exemplary hip spacer, per an embodiment herein;

FIG. 4B shows a front-view illustration of an exemplary hip spacer, per an embodiment herein;

FIG. 4C shows a bottom-view illustration of an exemplary hip spacer, per an embodiment herein;

FIG. 4D shows a cross-sectioned front-view illustration of an exemplary hip spacer, per an embodiment herein;

FIG. 5A shows a front-left perspective illustration of an exemplary hip spacer system coupled to a femoral stem, per an embodiment herein;

FIG. 5B shows a front-view illustration of an exemplary hip spacer system coupled to a femoral stem, per an embodiment herein;

FIGS. 5C to 5K depict aspects of exemplary hip spacer systems, according to embodiments of the present invention;

FIG. 6 shows a front-view illustration of an exemplary knee antibiotic irrigation platform, per an embodiment herein;

FIG. 7 shows a front-view illustration of an exemplary knee antibiotic irrigation system installed about a human knee joint, per an embodiment herein;

FIG. 8A shows a front-top perspective illustration of an exemplary knee spacer system coupled to a femoral stem, per an embodiment herein;

FIG. 8B shows a front-top-left perspective view illustration of an exemplary knee spacer system coupled to a femoral stem, per an embodiment herein;

FIG. 9A shows a front-view illustration of an exemplary knee spacer, per an embodiment herein;

FIG. 9B shows a bottom cross-sectioned view illustration of an exemplary knee spacer, per an embodiment herein;

FIG. 9C shows a front cross-sectioned view illustration of an exemplary knee spacer, per an embodiment herein;

FIG. 10A shows a front-view illustration of an exemplary knee spacer disposed between a femoral implant and a tibial implant;

FIGS. 10B and 10C depict aspects of exemplary knee spacer systems, according to embodiments of the present invention;

FIG. 11 shows a front-top perspective illustration of an exemplary shoulder spacer system, per an embodiment herein;

FIG. 12A shows a back-view illustration of an exemplary shoulder spacer, per an embodiment herein;

FIG. 12B shows a left-view illustration of an exemplary shoulder spacer, per an embodiment herein;

FIG. 12C shows a front-view illustration of an exemplary shoulder spacer, per an embodiment herein;

FIGS. 12D and 12E depict aspects of exemplary shoulder spacer systems, according to embodiments of the present invention;

FIG. 13A shows a top-left perspective view illustration of a humeral head of an exemplary shoulder spacer, per an embodiment herein;

FIG. 13B shows a bottom-right perspective view illustration of a humeral head of an exemplary shoulder spacer, per an embodiment herein;

FIG. 13C shows a detailed left cross-sectioned view illustration of an inlet of a humeral head of an exemplary shoulder spacer, per an embodiment herein;

FIGS. 14A to 14D depict aspects of exemplary shoulder spacer systems, according to embodiments of the present invention;

FIG. 15 depicts aspects of exemplary shoulder spacer systems, according to embodiments of the present invention;

FIGS. 16A and 16B illustrate aspects of exemplary reverse shoulder spacer systems, according to embodiments of the present invention;

FIGS. 17A and 17B illustrate aspects of exemplary reverse shoulder spacer systems, according to embodiments of the present invention; and

FIG. 18 depicts aspects of exemplary reverse shoulder spacer systems, according to embodiments of the present invention.

FIG. 19 illustrates aspects of exemplary treatment methods, according to embodiments of the present invention.

### DETAILED DESCRIPTION

A number of surgical interventions are used to treat periprosthetic joint infection (PJI). The standard of care (SOC) for treatment of chronic PJI may include a two-stage exchange arthroplasty: surgical removal of the infected implant, aggressive debridement and two-stage exchange arthroplasty with administration of systemic antibiotics. Stage 1 of the procedure includes removal of the infected prosthesis and implantation of a temporary antibiotic impregnated cement spacer with adjuvant administration of systemic antibiotic therapy as needed, typically for a period of at least 6 weeks. Stage 2 of the procedure is performed when patients are considered infection free and includes removal of the temporary spacer and implantation of a new permanent prosthesis. Irrigation, and irrigation and debridement with implant retention, is an attractive alternative due to decreased patient morbidity associated with resection arthroplasty.

Prosthesis retention is an attractive alternative due to overall decreased patient morbidity. For acute cases, select patients retain their implants as the biofilm does not have time to mature. Acute cases with well-fixed implants and a known pathogen can be treated with Debridement, Antibiotics and Implant Retention (DAIR). The DAIR procedure includes a radical debridement and irrigation of the infected joint, often with replacement of the modular implant components (i.e. knee: tibial polyethylene, hip: acetabular polyethylene liner and metal or ceramic femoral head).

Many factors influence the ultimate surgical management chosen for a given patient. Examples of these factors could include duration of symptoms, joint age (early, delayed, or late), infecting pathogen and its susceptibility pattern, prosthesis stability, and the patient's preexisting medical comorbidities. Other factors, such as the quality of the periprosthetic soft tissue, the options available for successful reconstructive surgery after resection arthroplasty, the expertise of the clinician(s), and the patient's preferences, also influence the surgical management. Clinical guidelines for PJI treatment have been published by the Infectious Diseases Society of America (IDSA). The guidelines state that implant retention strategies can be used in acute PJI where the biofilm does not have time to mature before surgical intervention.

Research shows the timeline for biofilm formation may be dependent on a number of host related factors and bacterial species, with animal studies showing biofilm can develop rapidly after bacterial exposure, in minutes to hours.

The potential for biofilm formation in minutes to hours may contribute to the limited success of implant retention treatments, as biofilm may already be attached to the retained implants even in acute cases, allowing continued dispersion of bacteria from the biofilm remaining after the irrigation and debridement procedure. Continuous local antibiotic irrigation delivered by systems and methods disclosed herein have the potential to eradicate any remaining biofilm on retained implants after irrigation and debridement, significantly improving success rate of implant retention strategies for treating PJI.

Many issues exist with current implant retention treatments. First, systemic antibiotic concentrations are not sufficient to eradicate biofilm. For example, when antibiotics are administered systemically, concentrations at the infection site are much lower than serum concentrations: local vancomycin HCl concentration is 10% to 60% and local tobramycin sulfate concentration is 9% to 13% of serum concentration. Achievement of locally therapeutic levels is crucial for clinical success; however, this is difficult due to the fact that most PJI pathogens are biofilm forming. Biofilm-encapsulated bacteria require minimum biofilm eradication concentrations (MBEC) of antibiotics that are several orders of magnitude (100 to 1000×) above the minimum inhibitory concentrations (MIC) sufficient to eradicate planktonic bacteria. Therapeutic target attainment at levels greater than the MBEC is impossible via systemic routes of administration without significant risk of toxicity to other organ systems.

Second, while current guidelines dictate 4-6 weeks of IV antibiotics and up to one year of oral antibiotics to eradicate the infection, long term antibiotic use increases development of antibiotic resistant organisms.

As such, provided herein are rapid and effective local infection therapy methods, systems, and devices that significantly reduce the mortality, morbidity, and the cost of care in rare musculoskeletal infections. Continuous delivery of antibiotic therapy locally, at the infection site, reduces edema and provides antibiotic irrigation, significantly improving outcomes while reducing the need for systemic antibiotics.

Such approaches can involve a spacer that is placed between permanent implants of a prosthesis, for a temporary period of time. In some cases, such permanent implants may not yet be infected between the implant and the bone, during the first 2-4 weeks following their implantation. Such implants remain well-fixed in bone. If a surgeon can detect infection early after its onset, the infection can be treated without removing the two permanent implants by placing a spacer therebetween temporarily, while temporarily irrigating the surrounding soft tissue to achieve certain clinical advantages. In such procedures, the surgeon may remove certain modular components of the prosthetic implant system, and replace them with an interpositional spacer as disclosed herein, for a period of time (e.g. having a duration within a range from about 3 days to about 3 weeks). Following the irrigation treatment period, the interpositional spacer can then be removed, and replaced with one or more permanent prosthetic replacement components.

Interpositional spacers (and related systems and methods) as disclosed herein are well suited for use in treating patients presenting with acute periprosthetic joint infection (PJI), for example as part of a Debridement, Antibiotics and Implant Retention (DAIR) procedure.

### Hip Spacer Platforms, Systems, and Devices

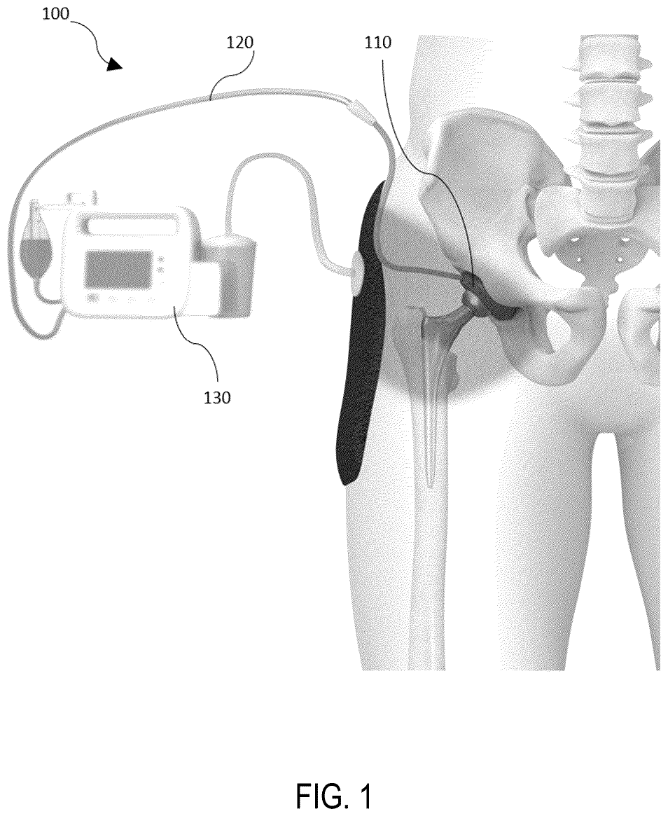

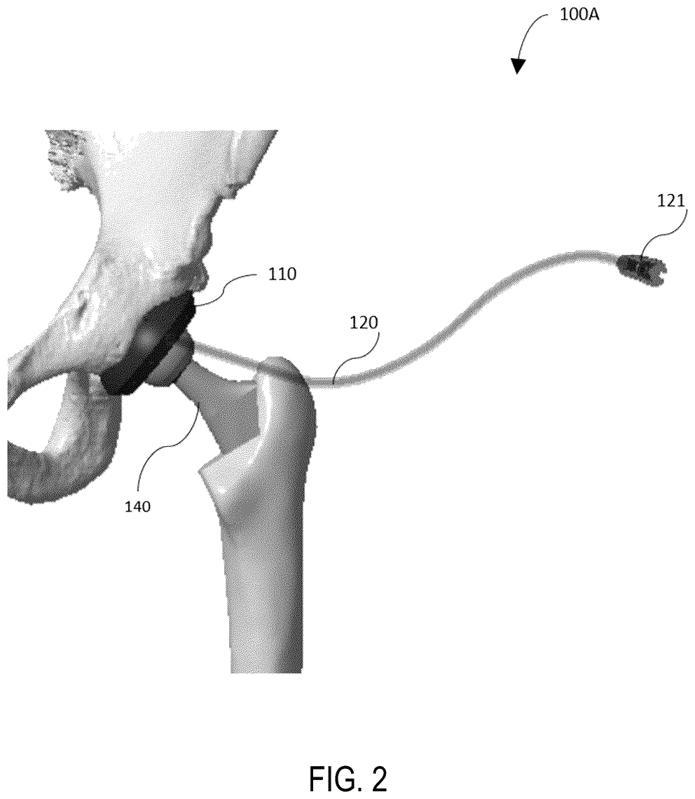

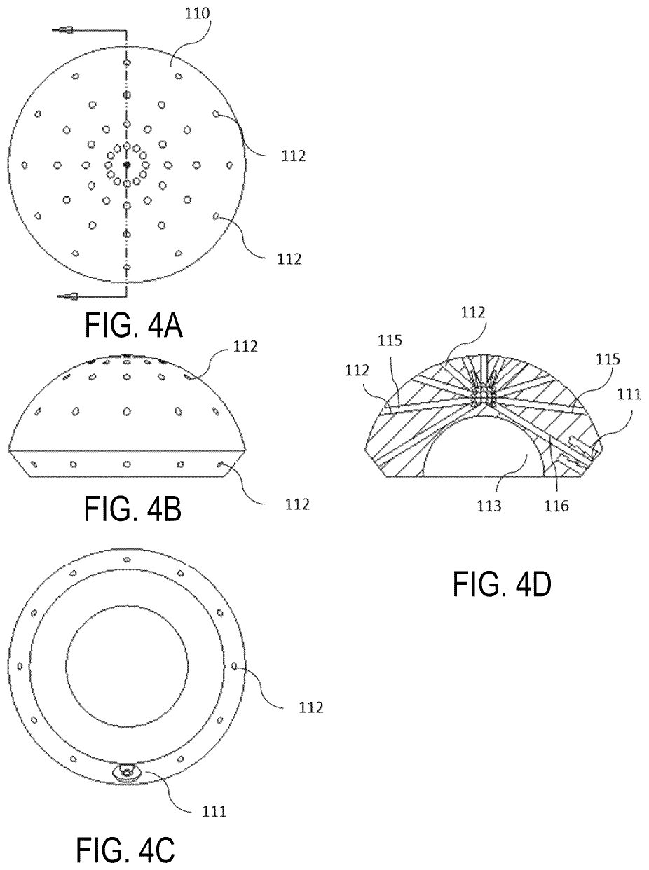

Provided herein, per FIG. 1, is a hip spacer platform 100 comprising a hip spacer system 100A (FIG. 2) and a negative pressure wound therapy pump 130. It is understood that the instant disclosure is not limited to a NPWT pump, and that any infusion and vacuum pump may be used. For example, embodiments encompass hip spacer platforms which include a vacuum pump with gravity infusion. In some embodiments, per FIGS. 2-5D, the hip spacer system 100A comprises a hip spacer 110 and a catheter 120. In some embodiments, per FIGS. 4A-4D, the hip spacer 110 comprises a fluid inlet 111, one or more fluid outlets 112, and a femoral head aperture 113. In some embodiments, the catheter 120 is in fluidic communication with the fluid inlet 111 of the hip spacer 110. In some embodiments, the negative pressure wound therapy (NPWT) pump 130 is in fluidic communication with the catheter 120 and the fluid outlet(s) 112 of the hip spacer 110. In some embodiments, the catheter 120 is coupled to the NPWT pump via a connection 121. Also provided herein is a hip spacing kit comprising two or more sizes of the hip spacer system 100A and the negative pressure wound therapy pump 130 (or any infusion and vacuum pump), so as to accommodate various patient anatomies. Hence, in some cases, a hip spacer platform or hip spacing kit may or may not include a NPWT pump.

As shown in FIGS. 4A-4D, in some embodiments, the hip spacer 110 comprises a single fluid inlet 111 and a plurality of fluid outlets 112. Alternatively, in some embodiments, the hip spacer 110 comprises 1, 2, 3, 4, 5, 6, 7, 8, 9, 10, or more fluid inlets 111. Alternatively, in some embodiments, the hip spacer 110 comprises 1, 2, 3, 4, 5, 6, 8, 10, 15, 20, 25, 30, 35, 40, 45, 50, 55, 60, 65, 70, 75, 80 or more fluid outlets 112. In some embodiments, an inner diameter of the fluid inlet 111 is equal to an inner diameter of the fluid outlet(s) 112. In some embodiments, an inner diameter of the fluid inlet 111 is greater than an inner diameter of the fluid outlet(s) 112. In some embodiments, an inner diameter of the fluid inlet 111 is less an inner diameter of the fluid outlet(s) 112. In some embodiments, the location of the fluid inlet 111 is specified to be closer to a standard incision location.

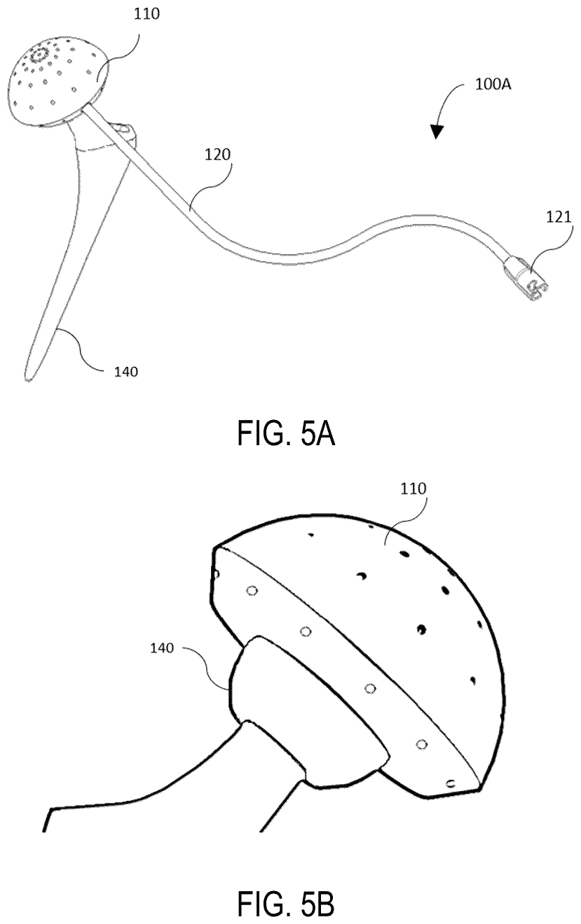

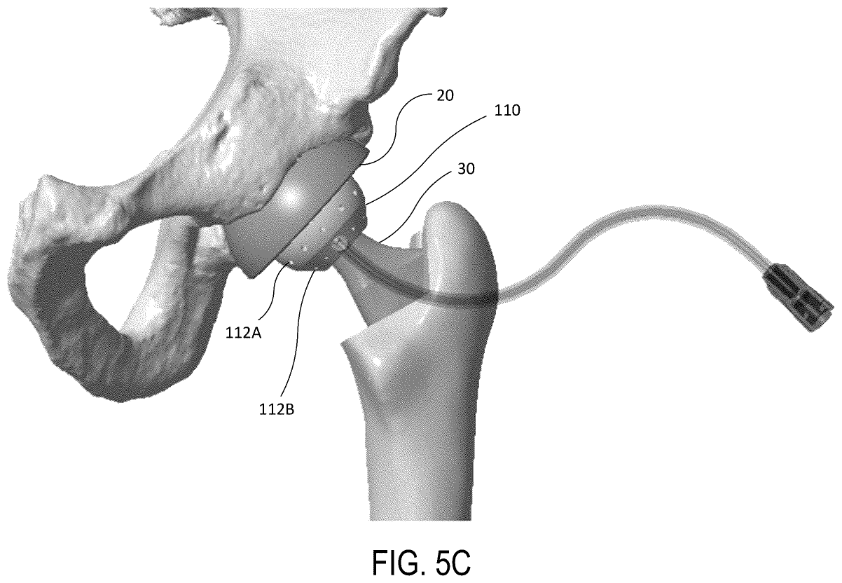

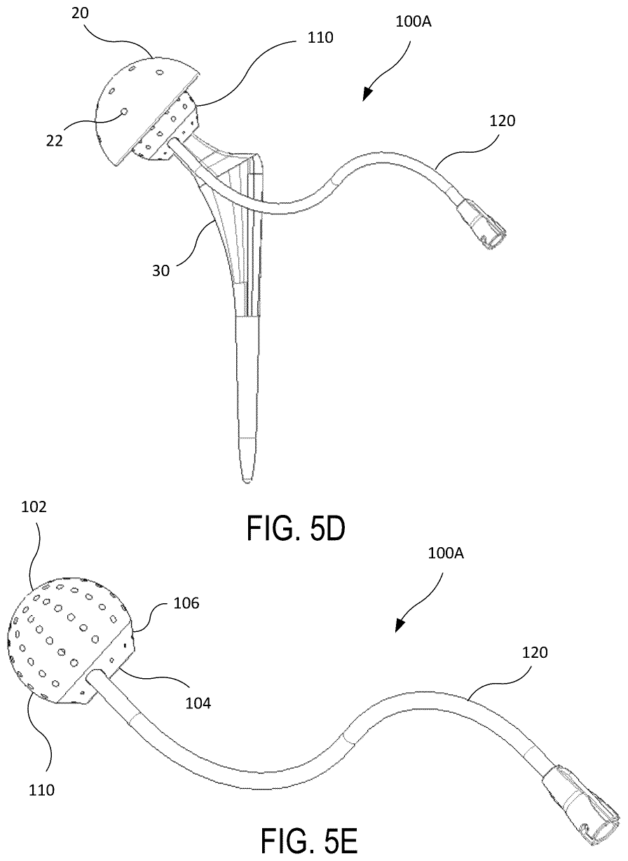

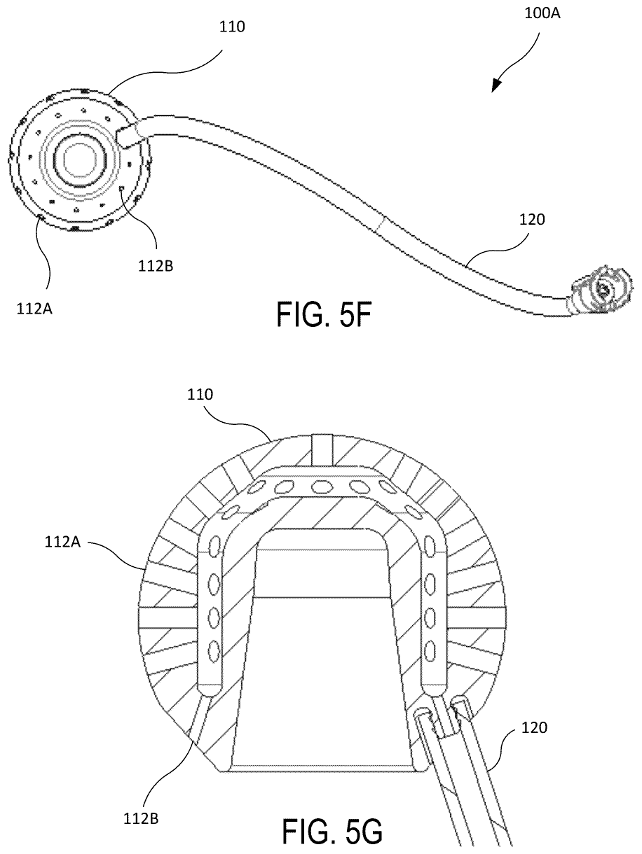

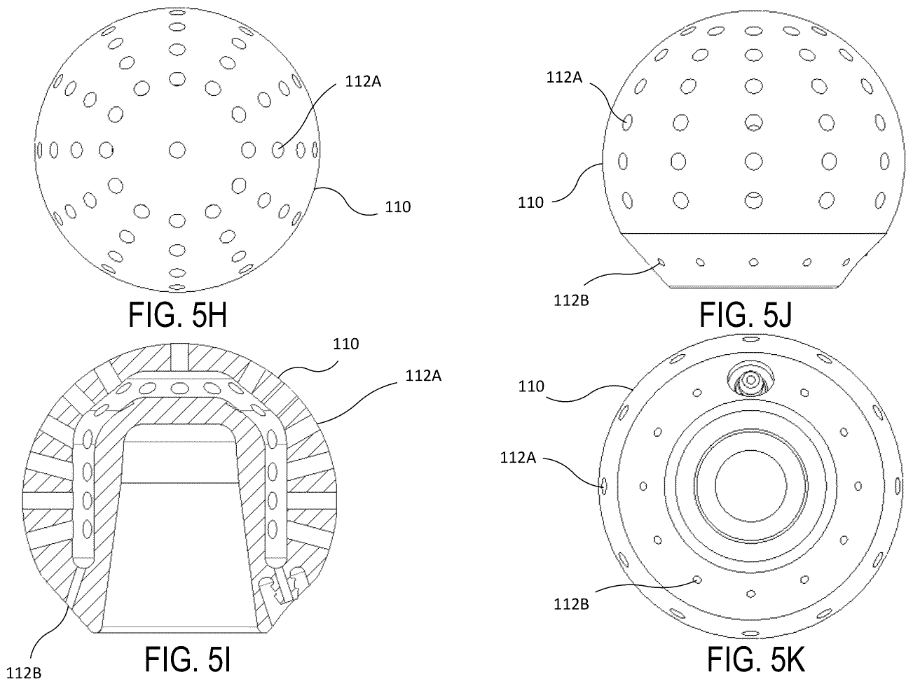

In some embodiments, as shown in FIG. 4D, the fluid inlet 111 is in fluidic communication with the fluid outlets 112 via an inlet channel 116 and one or more outlet channels 115. In some embodiments, each outlet channel 115 corresponds to a fluid outlet 112. In some embodiments, one or more outlet channels 115 each correspond to one or more fluid outlets 112. In some embodiments, the outlet channels 115 are configured in a polar array about a center of the hip spacer 110. In some embodiments, the fluid outlets 112 are arranged in a polar array about a center of the hip spacer 110. In some embodiments, the fluid outlets 112 are arranged in a polar array about a center of the hip spacer 110 in a series of radial and circular axes, wherein the radial axes intersect and wherein the circular axes are concentric. In some embodiments, the hip spacer 110 comprises 2, 4, 6, 8, 10, 12, 14, 16, 18, 20, 22, 24 or more radial axes. In some embodiments, the hip spacer 110 comprises 2, 4, 6, 8, 10, 12, 14, 16, 18, 20, 22, 24 or more circular axes. As shown, in some embodiments, the fluid outlets 112 are evenly arrayed about the hip spacer 110. Alternatively, the outlet channels 112 may be unevenly arrayed about the hip spacer 110. Alternatively, in some embodiments, the fluid outlets 112 are arranged in a rectilinear array. Further, in some embodiments, as shown, the inlet channel 116 and each of the outlet channels 115 intersect. In some embodiments, the inlet channel 116 and each of the outlet channels 115 intersect at a center axis of the hip spacer 110. In some embodiments, two or more of the fluid outlets 112 are concentric. In some embodiments, per FIGS. 4D, 5A and 5B, the hip spacer 110 mates with a retained femoral implant 140 (e.g., femoral stem in FIG. 5A, femoral head in FIG. 5B). In some embodiments, the femoral head aperture 113 of the hip spacer 110 mates with the retained femoral implant 140. In some embodiments, the hip spacer 110 articulates about the retained femoral implant 140. As shown, in some embodiments, the femoral head aperture 113 has a concave hemispherical shape. In some embodiments, the hip spacer 110 is disposed about a femoral head. Further as shown, the hip spacer 110 comprises a single uniform body. Alternatively, in some embodiments, the hip spacer 110 comprises two or more bodies that are removably coupled. Alternatively, in some embodiments, the hip spacer 110 comprises two or more components that are removably coupled, wherein the femoral head aperture 113 is multi-hemispherical. In some embodiments, at least a portion of an outer surface of the hip spacer 110 is hemispherical.

With continuing reference to FIG. 4D, it is understood that in some embodiments the total volume defined by the outlet channels 115 is less than the volume of a bolus or discrete amount of medicament or fluid delivered therethrough (which may be part of a periodic delivery regimen). Relatedly, the total volume defined by the outlet channels 115 can be minimized so that antibiotics or other fluids administered to the patient via the spacer are not retained by the spacer, and hence the spacer does not operate as a reservoir. This approach can maximize the amount of fluid that is delivered or exiting the spacer, and minimize the amount of fluid that is retained within the spacer. Hence, for example, if 50 cc of fluid is provided to the spacer, the total volume defined by the outlet channels can be less than 50 cc. This principle regarding total volume of outlet channels can also apply to knee spacer and/or shoulder spacer embodiments disclosed herein.

In some embodiments, the catheter is removably coupled to the fluid inlet 111 of the hip spacer 110. In some embodiments, the fluid inlet 111 of the hip spacer 110 comprises a luer taper, a barb fitting, or both to removably couple to the catheter. In some embodiments, the fluid inlet 111 of the hip spacer 110 is configured to couple to the catheter intra-operatively. In some embodiments, the catheter is permanently coupled to the fluid inlet 111 of the hip spacer 110. In some embodiments, the hip spacer system 100A further comprises a sterile packaging enclosing the hip spacer 110. In some embodiments, the sterile packaging comprises a double sterile barrier for introduction into the sterile field.

In some embodiments, the hip spacer platform 100 is configured to be implanted during a short-period of time. In some embodiments, the hip spacer 110 provides local irrigation, drug administration, or both. In some embodiments, the fluid inlet 111 and the fluid outlet(s) 112 of the hip spacer 110 provide local irrigation, drug administration, or both. In some embodiments, the hip spacer 110 is made of a biocompatible polymer. In some embodiments, the biocompatible polymer is a synthetic polymer. In some embodiments, the synthetic polymer is low-density polyethylene (LDPE), high-density polyethylene (HDPE), polypropylene (PP), polyvinyl chloride (PVC) polystyrene (PS) nylon, polytetrafluoroethylene, or a thermoplastic polyurethane (TPU). In some cases, a biocompatible polymer is acetyl copolymer (e.g. Delrin® or Celcon®), silicone, polyether ether ketone (PEEK), a polyurethane, including a flexible polyurethane, a biocompatible elastomer, or a ultrahigh molecular weight polyethylene (UHMWPE). In some embodiments, the hip spacer 110 is at least partially rigid. In some embodiments, the hip spacer 110 is at least partially flexible. In some embodiments, at least a portion of the hip spacer 110 has a modulus of elasticity of about 1 GPa to about 300 GPa.

In some embodiments, the hip spacer is configured to be positioned between the femoral head and acetabular cup of a permanent hip prosthesis. In some embodiments, the hip spacer is configured to be positioned between the femoral stem and acetabular cup of a permanent hip prosthesis with the prosthetic femoral removed. In both cases, the hip spacer has an additional function of protecting the surfaces of the prosthetic components during irrigation or treatment.

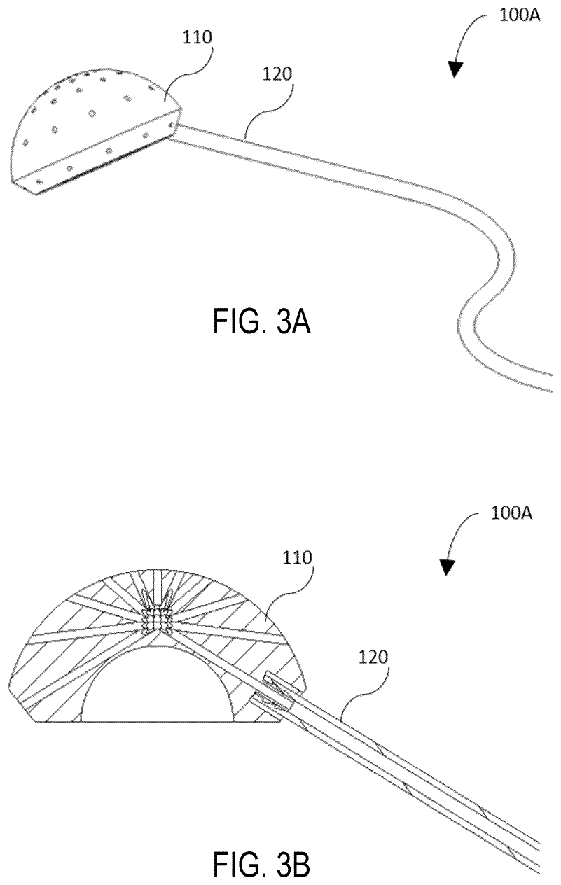

As depicted in FIG. 5C, a hip spacer 110 can be placed between an acetabular cup 20 and a femoral stem 30 of a permanent hip prosthesis. In some instances, the acetabular cup 20 and/or the femoral stem can include or be fabricated from titanium. Fluid outlets may be positioned all over the hip spacer. For example, fluid outlets 112A may be on the spacer surface that contacts the cup 20. In this way, antibiotic or other materials can be provided into the acetabular cup 20, which itself may have holes in it, so as to facilitate the flow or delivery of antibiotic or other materials to acetabular bone. Fluid outlets 112B may be on the exposed surface of the spacer 110. Outlets 112B can facilitate the flow of treatment fluid to joint space between the first and second implants (20, 30). Hip spacer 110 can also include outlets which facilitate the flow of treatment fluid to spaces between the spacer and the second implant 30, and such flow may also be facilitated by the presence of fluted channels on the underside of the spacer. In the embodiment depicted in FIG. 5C, the hip spacer 110 can be used in a situation where the original prosthetic included a metal cup or shell which is attached with the acetabular bone, a liner (e.g. plastic) which is disposed within the metal cup or shell, a spherical head which articulates against or otherwise engages the liner, and stem or trunnion which is coupled with the spherical head or ball, and the surgical procedure involves removing the liner and spherical head, and temporarily inserting therefore the hip spacer 110. In contrast, the hip spacer 110 depicted in FIG. 3A (which is shown as less than hemispherical) can be used when the stem and spherical head of the original prosthetic are retained, and only the liner or liner and cup are removed.

As shown in FIGS. 5D and 5E, a hip spacer system 100A can include a hip spacer 110 and a catheter 120. The hip spacer 100 can be coupled with a retained femoral stem 30 and acetabular cup 20 of a permanent hip prosthesis. As shown here, acetabular cup 20 includes one or more fluid outlets or apertures 22 whereby fluid provided via the spacer 110 can flow therethrough and toward acetabular bone. Spacer 100 includes a first surface 102 configured for articulating engagement with the first implant 20, a second surface 104 configured for fixed engagement with the second implant 30, and an exposed surface 106 disposed between the first surface 102 and the second surface 104. As shown in FIG. 5F, hip spacer system 100A can include a hip spacer 110 and a catheter 120, and as shown in the cross-section view of FIG. 5G, surfaces of the spacer 110 can include apertures 112A and 112B through which treatment fluid may flow. As shown in the top view of FIG. 5H, the cross-section view of FIG. 5I, the side view of FIG. 5J, and/or the bottom view of FIG. 5K, a hip spacer 110 can include apertures 112A and 112B through which treatment fluid may flow.

### Knee Spacer Platforms, Systems, and Devices

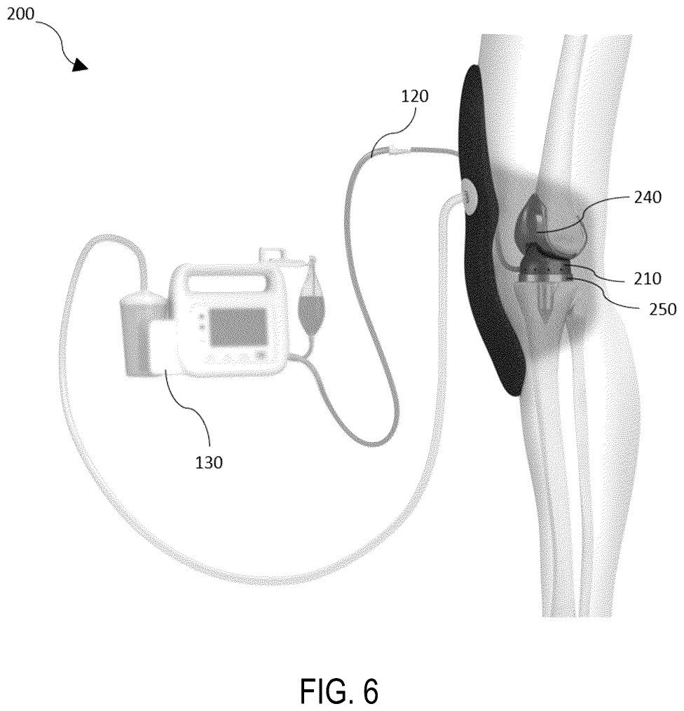

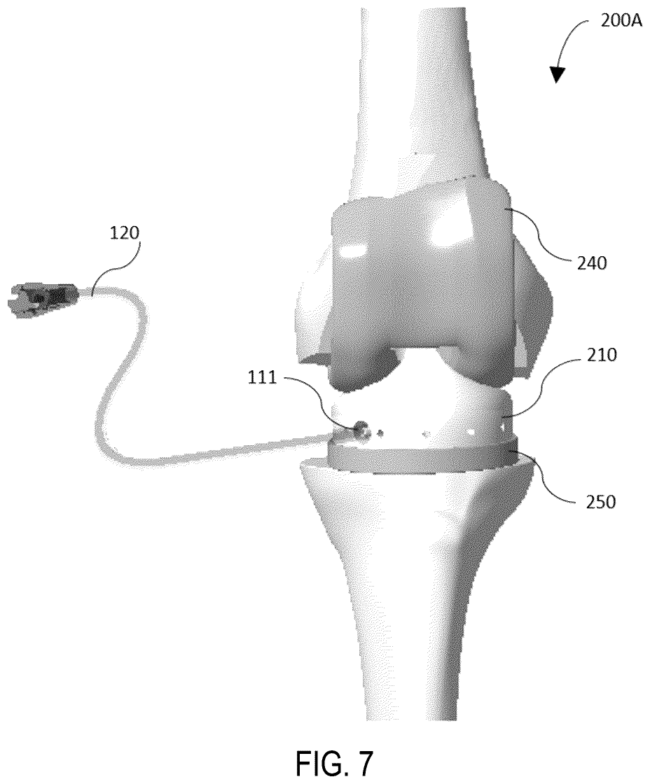

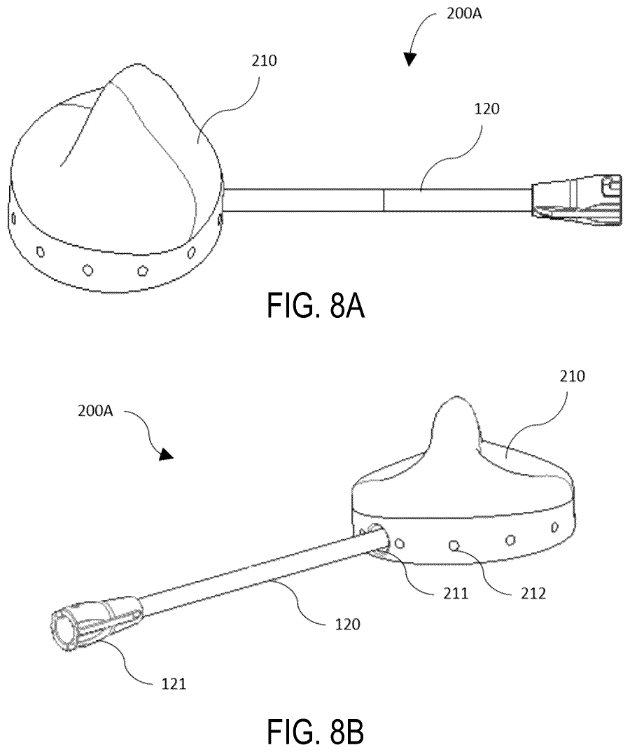

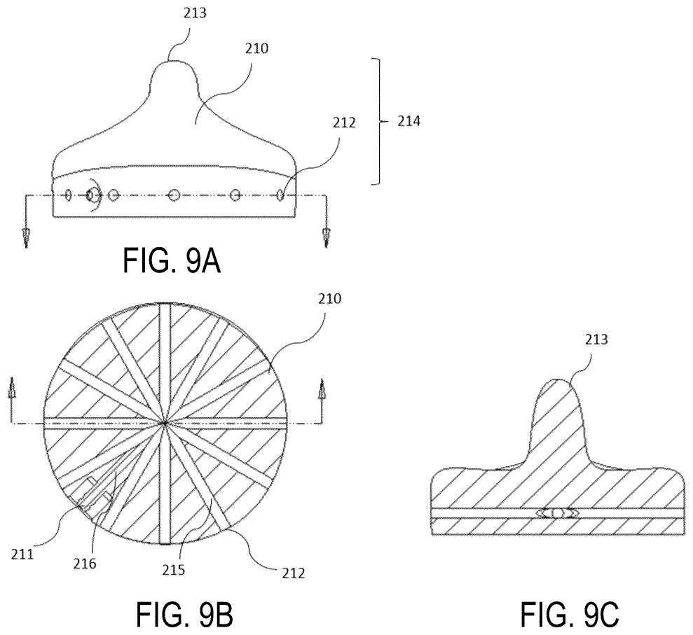

Provided herein, per FIG. 6, is a knee spacer platform 200 comprising a knee spacer system 200A (FIG. 7) and a negative pressure wound therapy pump 130. It is understood that the instant disclosure is not limited to a NPWT pump, and that any infusion and vacuum pump may be used. For example, embodiments encompass knee spacer platforms which include a vacuum pump with gravity infusion. In some embodiments, per FIGS. 7-8B, the knee spacer system 200A comprises a knee spacer 210 and a catheter 120. In some embodiments, per FIGS. 8A-9C, the knee spacer 210 comprises a fluid inlet 211, one or more fluid outlets 212, and a femoral component post 213. In some embodiments, the catheter 120 is in fluidic communication with the fluid inlet 211 of the knee spacer 210. In some embodiments, the negative pressure wound therapy pump 130 is in fluidic communication with the catheter 120 and the fluid outlet(s) 212 of the knee spacer 210. In some embodiments, the catheter 120 is coupled to the NPWT pump via a connection 121. Also provided herein is a knee spacing kit comprising two or more sizes of the knee spacer system and the negative pressure wound therapy pump (or any infusion and vacuum pump), so as to accommodate various patient anatomies. Hence, in some cases, a knee spacer platform or knee spacing kit may or may not include a NPWT pump.

Provided herein, per FIG. 6, is a knee spacer platform 200 comprising a knee spacer system 200A and a negative pressure wound therapy pump 130. In some embodiments, per FIGS. 7-8B, the knee spacer system 200A comprises a knee spacer 210 and a catheter 120. In some embodiments, per FIGS. 9A-9C, the knee spacer 210 comprises a fluid inlet 211, a fluid outlet 212, and a femoral component post 213. In some embodiments, the catheter 120 is in fluidic communication with the fluid inlet 211 of the knee spacer 210. In some embodiments, the negative pressure wound therapy pump 130 is in fluidic communication with the catheter 120 and the fluid outlet 212 of the knee spacer 210. In some embodiments, the location of the fluid inlet 211 is specified to be closer to a standard incision location.

As shown in FIGS. 9A-9C, the knee spacer 210 comprises a plurality of fluid outlets 212 arranged in a polar array arranged about a center of the knee spacer 210. In some embodiments, the knee spacer 210 comprises 2, 4, 6, 8, 10, 12, 14, 16, 18, 20, 22, 24 or more fluid outlets 212. Further, as shown, in some embodiments, the fluid inlet 211 is arranged between two consecutive fluid outlets 212. Alternatively, in some embodiments, the fluid outlets 212 are arranged in a rectilinear array. In some embodiments, an inner diameter of the fluid inlet 211 is equal to an inner diameter of the fluid outlet(s) 212. In some embodiments, an inner diameter of the fluid inlet 211 is greater than an inner diameter of the fluid outlet(s) 212. In some embodiments, an inner diameter of the fluid inlet 211 is less an inner diameter of the fluid outlet(s) 212. In some embodiments, as shown in FIG. 9B, the fluid inlet 211 is in fluidic communication with the fluid outlets 212 via an inlet channel 216 and one or more outlet channels 215. In some embodiments, each outlet channel 215 corresponds to a fluid outlet 212. In some embodiments, one or more outlet channels 215 each correspond to one or more fluid outlets 212. In some embodiments, the inlet channel 216 and each of the outlet channels 215 intersect. In some embodiments, the inlet channel 216 and each of the outlet channels 215 intersect at a center axis of the knee spacer 210.

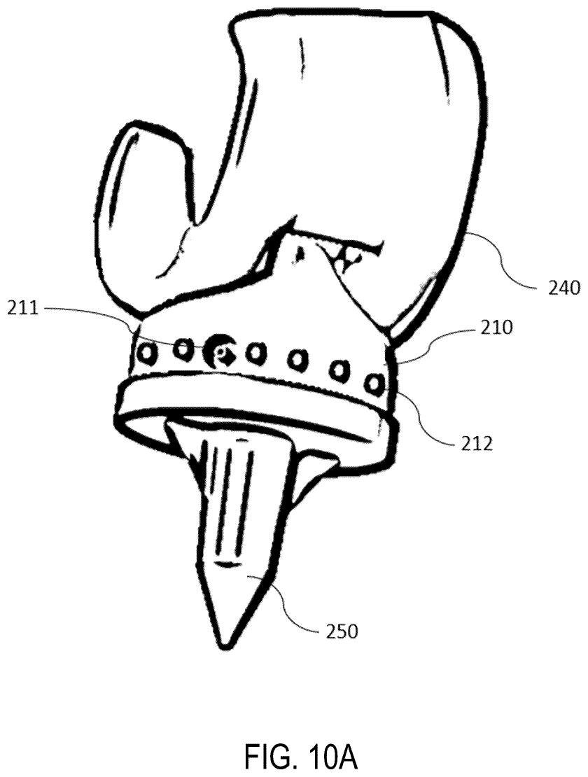

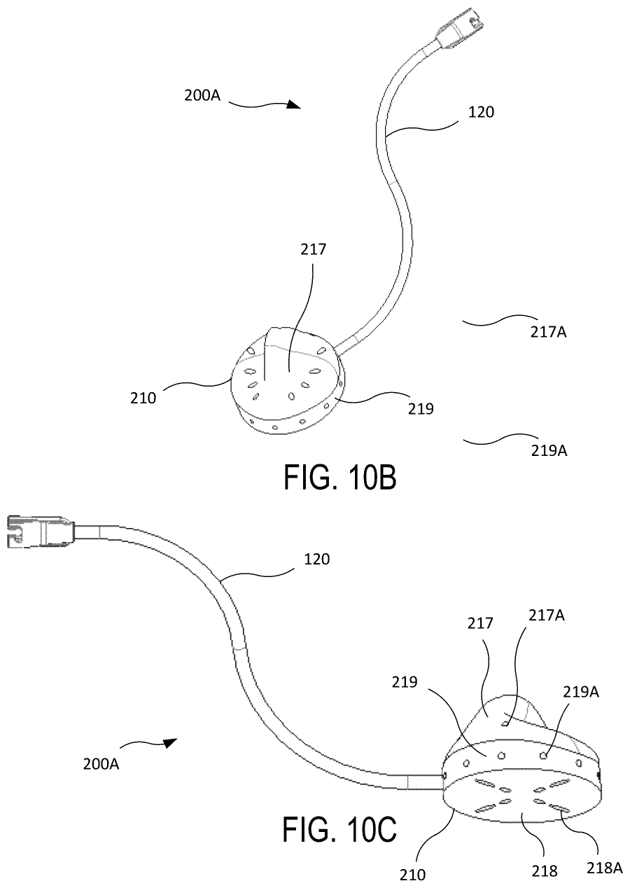

In some embodiments, per FIG. 10, the knee spacer 210 mates with a femoral component 240 and a tibial tray 250. In some embodiments, a femoral component post 213 of the knee spacer 210 mates with the femoral component 240. In some embodiments, the knee spacer 210 articulates about the retained femoral component 240. As shown, in some embodiments, the femoral component post 213 is symmetric about a plane. Further as shown in FIG. 9A, in some embodiments, a first surface 214 of the femoral component post 213 has a rounded outer edge. In some embodiments, the first surface 214 of the femoral component post 213 comprises a single uninterrupted surface. Further as shown, the knee spacer 210 comprises a single uniform body. Alternatively, in some embodiments, the knee spacer 210 comprises two or more bodies that are removably coupled.

In some embodiments, the catheter is removably coupled to the fluid inlet 211 of the knee spacer 210. In some embodiments, the fluid inlet 211 of the knee spacer 210 comprises a luer taper, a barb fitting, or both to removably couple to the catheter. In some embodiments, the fluid inlet 211 of the knee spacer 210 is configured to couple to the catheter intra-operatively. In some embodiments, the catheter is permanently coupled to the fluid inlet 211 of the knee spacer 210. In some embodiments, the knee spacer system 200A further comprises a sterile packaging enclosing the knee spacer 210. In some embodiments, the sterile packaging comprises a double sterile barrier for introduction into the sterile field.

In some embodiments, the knee spacer platform 200 is configured to be implanted during a short-period of time. In some embodiments, the knee spacer 210 provides local irrigation, drug administration, or both. In some embodiments, the fluid inlet 211 and the fluid outlet(s) 212 of the knee spacer 210 provide local irrigation, drug administration, or both. In some embodiments, the knee spacer 210 is made of a biocompatible polymer. In some embodiments, the biocompatible polymer is a synthetic polymer. In some embodiments, the synthetic polymer is low-density polyethylene (LDPE), high-density polyethylene (HDPE), polypropylene (PP), polyvinyl chloride (PVC) polystyrene (PS) nylon, polytetrafluoroethylene, or a thermoplastic polyurethane (TPU). In some cases, a biocompatible polymer is acetyl copolymer (e.g. Delrin® or Celcon®), silicone, polyether ether ketone (PEEK), a polyurethane, including a flexible polyurethane, a biocompatible elastomer, or a ultrahigh molecular weight polyethylene (UHMWPE). In some embodiments, the knee spacer 210 is at least partially rigid. In some embodiments, the knee spacer 210 is at least partially flexible. In some embodiments, at least a portion of the knee spacer 210 has a modulus of elasticity of about 1 GPa to about 300 GPa.

In some embodiments, the knee spacer is configured to be positioned between the tibial and femoral components of a permanent knee prosthesis. The knee spacer has an additional function of protecting the surfaces of the prosthetic components during irrigation or treatment.

As shown in FIGS. 10B and 10C, a knee spacer system 200A can include a knee spacer 210 and a catheter 120. The knee spacer 210 can be coupled with a retained femoral component and tibial component of a permanent knee prosthesis. In some cases, the femoral component can include one or more fluid outlets or apertures whereby fluid provided via the spacer 210 can flow therethrough and toward femoral bone. In some cases, the tibial component can include one or more fluid outlets or apertures whereby fluid provided via the spacer 210 can flow therethrough and toward tibial bone. Spacer 210 includes a first surface 217 configured for articulating engagement with a first implant (e.g. femoral implant), a second surface 218 configured for fixed engagement with a second implant (e.g. tibial implant), and an exposed surface 219 disposed between the first surface 217 and the second surface 218. A knee spacer 210 can include outlets through which treatment fluid may flow. For example, first surface 217 can include outlets 217A through which treatment fluid may flow toward a femoral implant, second surface 218 can include outlets 218A through which treatment fluid may flow toward a tibial implant, and exposed surface 219 can include outlets 219A through which treatment fluid may flow into a joint space between the femoral implant and the tibial implant, for example to treat the nearby soft tissue.

### Shoulder Spacer Platforms, Systems, and Devices

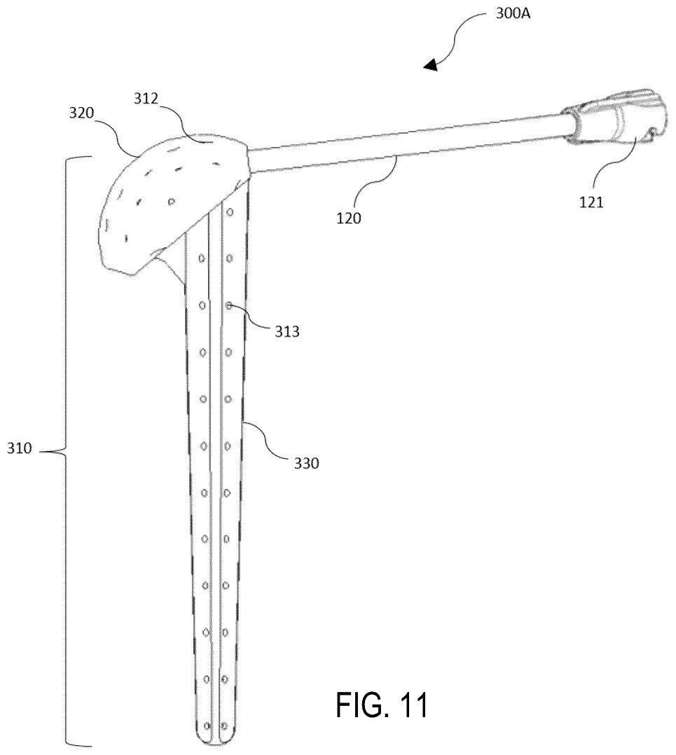

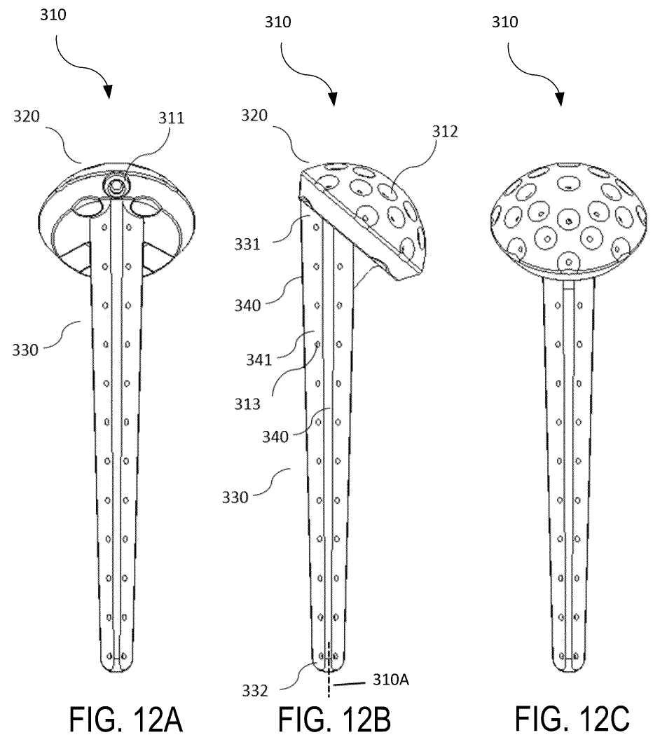

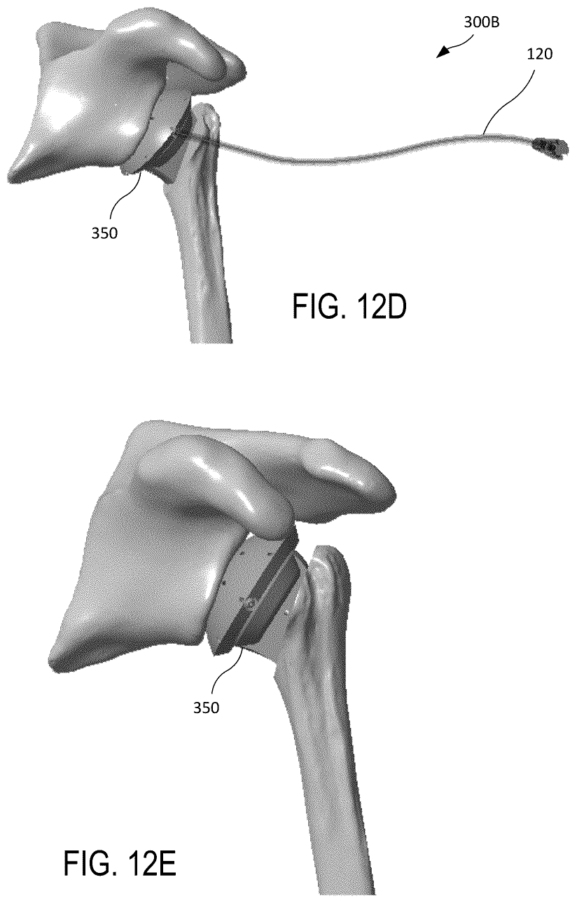

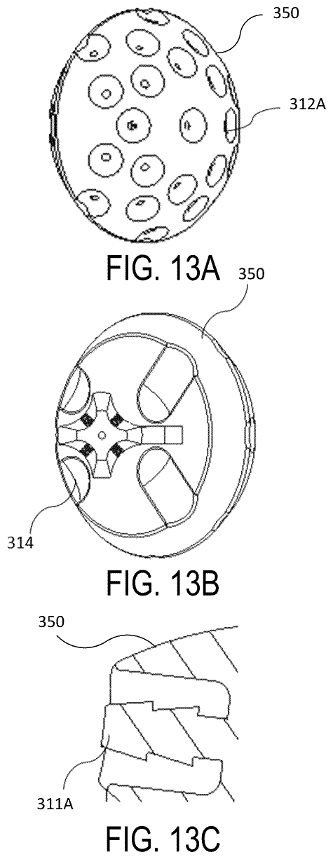

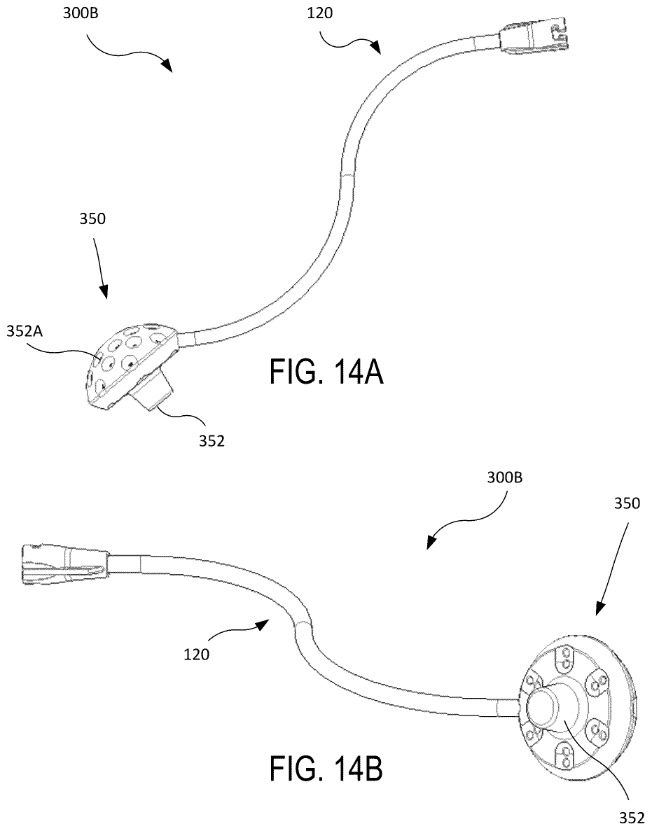

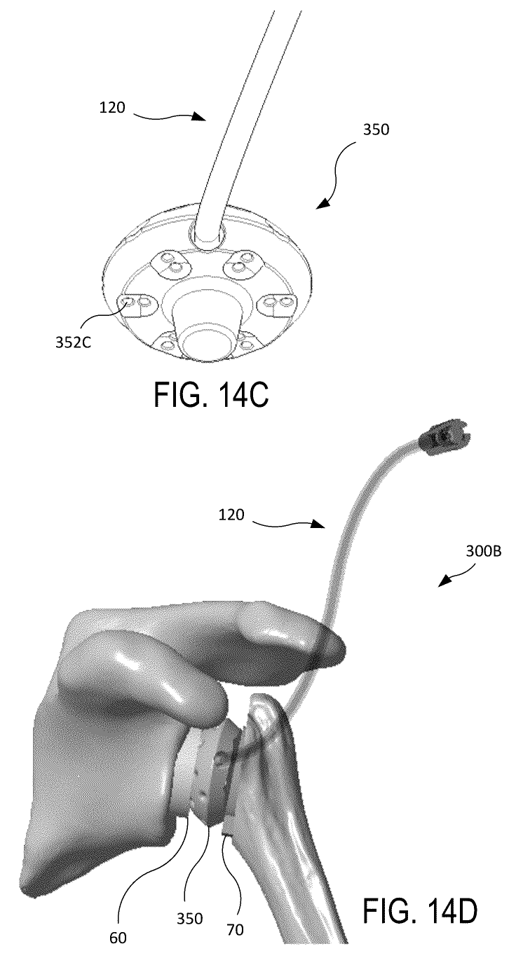

Provided herein, is a shoulder spacer platform comprising a shoulder spacer system (e.g. 300A or 300B) and a negative pressure wound therapy pump as described herein 130. It is understood that the instant disclosure is not limited to a NPWT pump, and that any infusion and vacuum pump may be used. For example, embodiments encompass shoulder spacer platforms which include a vacuum pump with gravity infusion. In some embodiments, per FIG. 11, the shoulder spacer system 300A comprises a shoulder spacer 310 and a catheter 120. In contrast to other embodiments disclosed herein involving an interpositional spacer (e.g. for placement between two implanted prosthetic components) such as hip spacer 110 shown in FIG. 3A, knee spacer 210 shown in FIG. 8B, and shoulder spacer 350 shown in FIG. 13A, it can be seen that shoulder spacer 310 of FIG. 11 would not be placed between two implanted prosthetics (e.g. between a humeral stem and a glenoid component of a shoulder prosthesis), because shoulder spacer 310 includes humeral stem 330. In some embodiments, the humeral head 320 is removably coupled to the humeral stem 330. In some embodiments, the humeral head 320 is coupled to the humeral stem 330 via a tapered connection, a dovetail connection, a threaded connection, a flanged connection, a snap buckle, a winged nut, or any other coupling mechanism. In some embodiments, the coupling mechanism allows for offset adjustments. Such embodiments allow for reduced inventory and better patient matching. According to some embodiments, shoulder spacer 310 depicted in FIG. 11 can be used to replace a permanent shoulder prosthetic implant. According to some embodiments, shoulder spacer 310 depicted in FIGS. 12A to 12C can be used to replace a permanent shoulder prosthetic implant. In some cases, shoulder spacer 310 can be used in conjunction with a permanent glenoid prosthetic implant, such as implant 60 depicted in FIG. 14D.

It is appreciated that shoulder spacer 310 or shoulder spacer system 300A can include any one or more features of the devices or can be configured to perform any one or more of the treatment method aspects of those disclosed in U.S. Provisional Patent Application Nos. 61/208,540 filed Feb. 25, 2009 or 62/180,986 filed Jun. 17, 2015, or in U.S. Patent Publication Nos. 2010/0217401 or 2016/0367371. The content of each of these filings is incorporated herein by reference.

In some embodiments, the shoulder spacer 310 comprises a fluid inlet 311 as shown in FIG. 12A. Likewise, a shoulder spacer 350 as shown in FIG. 13C can include a fluid inlet 311A. In some embodiments, the shoulder spacer 310 comprises one or more head fluid outlets 312. Likewise, a shoulder spacer 350 as shown in FIG. 13B can include one or more fluid outlets 312A. In some embodiments, the catheter 120 is in fluidic communication with the fluid inlet 311 of the shoulder spacer 310 (or with inlet 311A of shoulder spacer 350). In some embodiments, the negative pressure wound therapy pump 130 is in fluidic communication with the catheter 120, and the fluid outlet(s) 313. In some embodiments, the catheter 120 is coupled to the NPWT pump via a connection 121. In some embodiments, the location of the fluid inlet 311 (or 311A) is specified to be closer to a standard incision location. Also provided herein is a shoulder spacing kit comprising two or more sizes of the shoulder spacer system and the negative pressure wound therapy pump (or any infusion and vacuum pump), so as to accommodate various patient anatomies. Hence, in some cases, a shoulder spacer platform or shoulder spacing kit may or may not include a NPWT pump.

As shown in FIGS. 12A-13C, a humeral head or surface (e.g. of a shoulder spacer 310 depicted in FIGS. 11-12C or an interpositional shoulder spacer 350 depicted in FIGS. 12D-13B) can include a plurality of fluid outlets 312 or 312A, respectively, arranged in a polar array about a center of the humeral head 320 or spacer 350, respectively. In some embodiments, the humeral head 320 or shoulder spacer 350 comprises 2, 4, 6, 8, 10, 12, 14, 16, 18, 20, 22, 24 or more fluid outlets 312 or 312A, respectively. Alternatively, in some embodiments, the fluid outlets 312 or 312A are arranged in a rectilinear array. In some embodiments, an inner diameter of the fluid inlet 311 or 311A is equal to an inner diameter of the fluid outlet(s) 312 or 312A, respectively. In some embodiments, an inner diameter of the fluid inlet 311 or 311A is greater than an inner diameter of the fluid outlet(s) 312 or 312A, respectively. In some embodiments, an inner diameter of the fluid inlet 311 or 311A is less than an inner diameter of the fluid outlet 312(s) or 312A(s), respectively. In some embodiments, the fluid inlet 311 or 311A is in fluidic communication with the fluid outlets 312 or 312A, respectively, via an inlet channel and one or more outlet channels. In some embodiments, each outlet channel corresponds to a fluid outlet 312 or 312A. In some embodiments, one or more outlet channels each correspond to one or more fluid outlets 312 or 312A. In some embodiments, the inlet channel and each of the outlet channels intersect. In some embodiments, the inlet channel and each of the outlet channels intersect at a center axis of the shoulder spacer 310 (or stem thereof) or shoulder spacer 350.

In some embodiments, an interpositional shoulder spacer 350 comprises one or more fluted channels 314 as depicted in FIG. 13B. In some embodiments, the one or more fluted channels 314 are arrayed about a center axis of the shoulder spacer 350. In some embodiments, the shoulder spacer 350 comprises 1, 2, 3, 4, 5, 6, 7, 8, 9, 10, or more fluted channels 314 arrayed about the shoulder spacer 350. In some embodiments, one of more of the fluted channels 314 may include one or more fluid outlets. In some embodiments, the fluted channels 314 enable fluid flow under the shoulder spacer 350, between the shoulder spacer 350 and the humeral stem. In some embodiments, the shoulder spacer 350 is undersized to allow fluid flow thereover. It is understood that shoulder spacer 310 may include one or more similar fluted channels having one or more properties of the fluted channels 314 described here. It is also understood that hip spacer 110 and knee spacer 210 may include one or more similar fluted channels having one or more properties of the fluted channels 314 described here.

As shown in FIGS. 12A-C, in some embodiments, the humeral stem 330 comprises an elongate body having a longitudinal axis 310A, wherein the humeral stem 330 comprises a first end 331 and a second end 332 opposite the first end. The second, or distal, end 332 may be disposed in the medullary canal of a bone. In some embodiments, the humeral stem 330 can vary in size and/or longitudinal length. In some embodiments, the humeral stem 330 comprises a stem channel extending between the first end 331 and the second end 332. In some embodiments, the stem channel is configured to deliver fluid to the medullary canal via the one or more stem fluid outlets 313.

In some embodiments, the humeral stem 330 comprises a plurality of protrusions 340, protruding radially outward from a center axis of the humeral stem 330. The plurality of protrusions 340 may comprise any number of protrusions having any appropriate shape, size, or configuration to engage the medullary canal in a stable fashion. For example, the protrusions may comprise elongate fins extending along the longitudinal length of the humeral stem 330. In one exemplary embodiment, the plurality of protrusions 340 may comprise four fins, spaced equally at about 90° about the longitudinal axis 310A of the humeral stem 330. The plurality of protrusions 340 and the humeral stem 330 may be formed separately and coupled together. Alternatively or in combination, the plurality of protrusions 340 may be formed by removing material from the humeral stem 330, such that the plurality of protrusions and the humeral stem 330 are formed as a single member. Adjacent protrusions 340 may define one or more fluted regions 341 therebetween, wherein the fluted regions 341 are radially recessed compared to the protrusions 340. The fluted regions 341 may form a concave recessed region between adjacent protrusions 340.

The plurality of protrusions 340 and fluted regions 341 can be configured to minimize the surface area of the humeral stem 330 contacting the bone lining the medullary canal, such that the area of the bone flush with fluid being delivered via the fluid inlet 311 may be maximized. For example, the plurality of protrusions 340 and fluted regions 341 can be configured such that less than 50% of the surface area of the humeral stem 330 is in contact with the bone lining the medullary canal. Of course, this is not intended to be limiting and one of skill in the art will appreciate that any percentage of the surface area of the humeral stem 330 may contact the bone. In some embodiments, the humeral stem 330 comprises a plurality of identical fluted regions 341 defined by a plurality of elongate fins 340, distributed symmetrically about the longitudinal axis 310A of the humeral stem 330. Alternatively, a plurality of fluted regions 341 may be distributed asymmetrically about the longitudinal axis of the humeral stem 330, and/or may have different shapes or sizes.

In some embodiments, the humeral stem 330 comprises fluted regions 341 to allow for fluid flow in the medullary canal, allow for fluid flow between medullary canal and the joint space where a NPWT sponge will be.

In some embodiments, the plurality of stem fluid outlets 313 are in fluid communication with the internal channel of the humeral stem 330. The plurality of stem fluid outlets 313 may be configured to deliver the fluid, distributed through the humeral stem 330 internal channel, to the medullary canal, as well as adjacent tissue including the joint. The plurality of stem fluid outlets 313 may be disposed in one or more fluted regions 341, so as to deliver the fluid to the area of the bone not in contact with the humeral stem 330. The plurality of stem fluid outlets may comprise any number of outlet holes having any appropriate size, shape, or distribution. For example, the plurality of outlet holes may include a plurality of equally sized and spaced holes that extend axially along a line substantially parallel to the longitudinal axis 310A of the humeral stem 330. The plurality of outlet holes may be arranged in various configurations. The plurality of outlet holes may comprise holes having an identical shape and/or size, or holes having various shapes and/or sizes. Varying the hole size may allow further fluid control of the fluid as it exits different regions of the humeral stem 330.

The humeral stem 330 may be tapered to fit the medullary canal. For example, the humeral stem 330 and/or the plurality of protrusions 340 may be tapered from the first end 331 to the second end 332, as shown, so as to have a smaller radial cross-sectional area at the second end than at the first end. For example, the taper may comprise a gradual taper, wherein the extent of the taper may be preferably in a range from about 0.1° to about 10°, more preferably about 0.5° to about 5°, and even more preferably about 1° to about 5°, or about 1° to about 4°, or about 2° or about 3°. The taper may be adjusted to accommodate a medullary canal of a specific type of bone.

In some embodiments, the catheter is removably coupled to the fluid inlet 311 of the shoulder spacer 310. In some embodiments, the fluid inlet 311 of the shoulder spacer 310 comprises a luer taper, a barb fitting, or both to removably couple to the catheter. In some embodiments, the fluid inlet 311 of the shoulder spacer 310 is configured to couple to the catheter intra-operatively. In some embodiments, the catheter is permanently coupled to the fluid inlet 311 of the shoulder spacer 310. In some embodiments, the shoulder spacer system 300A further comprises a sterile packaging enclosing the shoulder spacer 310. In some embodiments, the sterile packaging comprises a double sterile barrier for introduction into the sterile field.

In some embodiments, the shoulder spacer platform is configured to be implanted during a short-period of time. In some embodiments, the shoulder spacer 310 provides local irrigation, drug administration, or both. In some embodiments, the fluid inlet 311 and the fluid outlet(s) 312 of the shoulder spacer 310 provide local irrigation, drug administration, or both. In some embodiments, the shoulder spacer 310 is made of a biocompatible polymer. In some embodiments, the biocompatible polymer is a synthetic polymer. In some embodiments, the synthetic polymer is low-density polyethylene (LDPE), high-density polyethylene (HDPE), polypropylene (PP), polyvinyl chloride (PVC) polystyrene (PS) nylon, polytetrafluoroethylene, or a thermoplastic polyurethane (TPU). In some cases, a biocompatible polymer is acetyl copolymer (e.g. Delrin® or Celcon®), silicone, polyether ether ketone (PEEK), a polyurethane, including a flexible polyurethane, a biocompatible elastomer, or a ultrahigh molecular weight polyethylene (UHMWPE). In some embodiments, the shoulder spacer 310 or shoulder spacer 350 is at least partially rigid. In some embodiments, the shoulder spacer 310 or shoulder spacer 350 is at least partially flexible. In some embodiments, at least a portion of the shoulder spacer 310 or shoulder spacer 350 has a modulus of elasticity of about 1 GPa to about 300 GPa.

In some embodiments, the shoulder spacing kit comprises two or more sizes of the shoulder spacer 310, two or more sizes of the humeral stem 330, or both. In some embodiments, the shoulder spacing kit comprises two or more sizes of the shoulder spacer 350.

In some embodiments, the shoulder spacer 350 is configured to be positioned between the humeral stem and glenoid components of a permanent shoulder prosthesis. The shoulder spacer has an additional function of protecting the surfaces of the prosthetic components during an irrigation or treatment period. In some embodiments, the shoulder spacer 310 is configured to be positioned adjacent a glenoid component of a permanent shoulder prosthesis.

FIG. 12D depicts shoulder spacer system 300A engaged with a glenoid bone and a humerus bone of a patient. Shoulder spacer system 300A includes a shoulder spacer 310 and a catheter 120. Similarly, FIG. 12E depicts shoulder spacer 350 engaged with a glenoid bone and a humerus bone of a patient.

In some embodiments, per FIGS. 14A-14D, the shoulder spacer system 300B comprises a shoulder spacer 350 and a catheter 120. It can be seen that shoulder spacer 350 includes a protrusion 352 which can engage a permanent prosthetic component, such as a humeral stem prosthetic component. A shoulder spacer 350 can be placed between a glenoid implant and a humeral implant of a permanent prosthesis. Fluid outlets may be positioned all over the shoulder spacer. For example, fluid outlets 352A may be on the spacer surface that contacts the glenoid implant prosthetic component. In this way, antibiotic or other materials can be provided into the glenoid implant 60, which itself may have holes in it, so as to facilitate the flow or delivery of antibiotic or other materials to glenoid bone. Fluid outlets may be on the exposed surface of the spacer 350. Such outlets can facilitate the flow of treatment fluid to joint space between the first and second implants. Shoulder spacer 350 can also include outlets 352C which facilitate the flow of treatment fluid to spaces between the spacer and the second implant (e.g. humeral stem 70), and such flow may also be facilitated by the presence of fluted channels on the underside of the spacer. In the embodiment depicted in FIG. 14D, the shoulder spacer 350 can be used in a situation where the original prosthetic (e.g. glenoid component 60) included a metal cup or shell which is attached with the glenoid bone, a liner (e.g. plastic) which is disposed within the metal cup or shell, a spherical head which articulates against or otherwise engages the liner, and stem or trunnion which is coupled with the spherical head or ball, and the surgical procedure involves removing the liner and spherical head, and temporarily inserting therefore the shoulder spacer 350 (e.g. while retaining the cup/shell of the glenoid component 60). In contrast, the shoulder spacer 350 depicted in FIG. 12D can be used when the stem and spherical head of the original prosthetic are retained, and only the liner or liner and cup are removed. The shoulder spacer 350 of FIGS. 12D and 14D may be the same, where shoulder spacer 350 of FIG. 12D is used when the glenoid prosthetic component is removed, and shoulder spacer 350 of FIG. 14D is used when the glenoid prosthetic component 60 is retained. In some cases, a shoulder spacer can be used when a spherical head of the humeral prosthetic implant is retained. In some cases, a shoulder spacer can be used when a spherical head of the humeral prosthetic implant is removed.

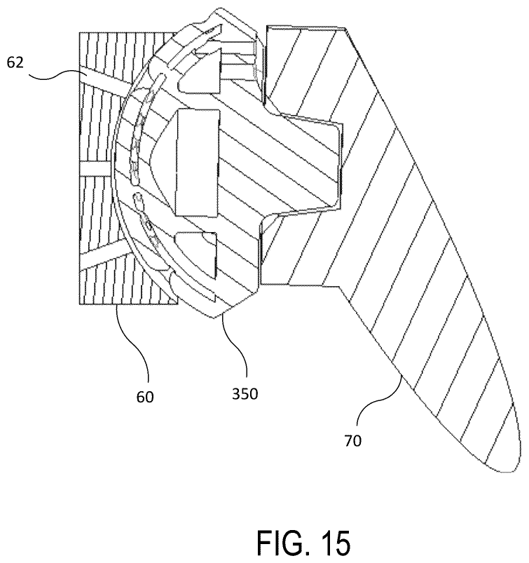

FIG. 15 depicts aspects of a shoulder spacer 350 according to embodiments of the present invention. As shown here, shoulder spacer 350 can be engaged with a glenoid implant component 60 and a humeral implant component 70 of a previously implanted prosthesis. As shown here, glenoid component 60 includes one or more holes or apertures 62 whereby fluid provided via the spacer 350 can flow therethrough and toward glenoid bone.

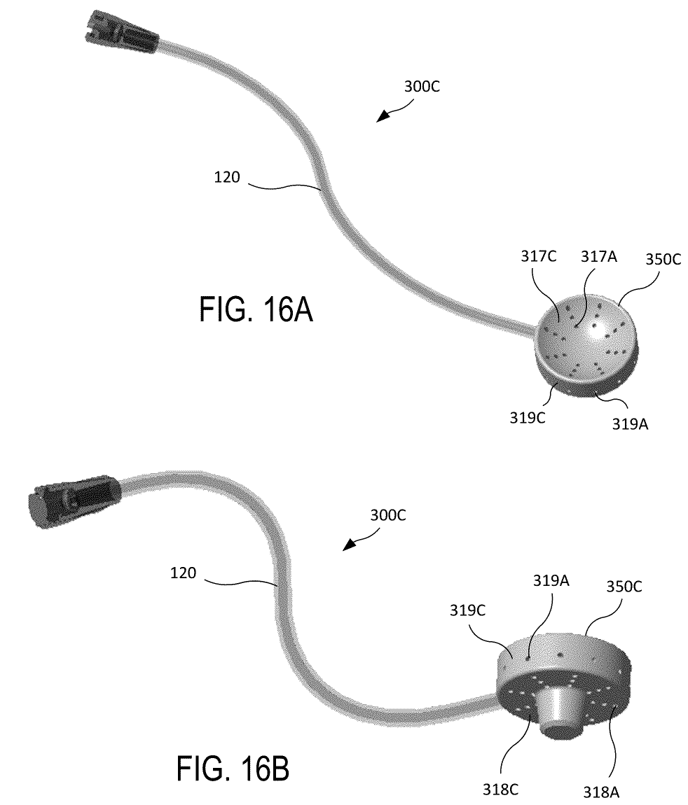

In addition to the anatomical shoulder spacer embodiments discussed above, embodiments of the present invention also encompass reverse shoulder spacer embodiments. For example, FIGS. 16A and 16B illustrate aspects of an exemplary reverse shoulder spacer system 300C. As depicted here, reverse shoulder spacer system 300C includes a reverse shoulder spacer 350C and a catheter 120. Reverse shoulder spacer 350C includes a first surface 317C configured for articulating engagement with a first implant (e.g. glenoid implant), a second surface 318C configured for fixed engagement with a second implant (e.g. humeral implant), and an exposed surface 319C disposed between the first surface 317C and the second surface 318C. A reverse shoulder spacer 350C can include outlets through which treatment fluid may flow. For example, first surface 317C can include outlets 317A through which treatment fluid may flow toward a glenoid implant, second surface 318C can include outlets 318A through which treatment fluid may flow toward a humeral implant, and exposed surface 319C can include outlets 319A through which treatment fluid may flow into a joint space between the glenoid implant and the humeral implant, for example to treat the nearby soft tissue.

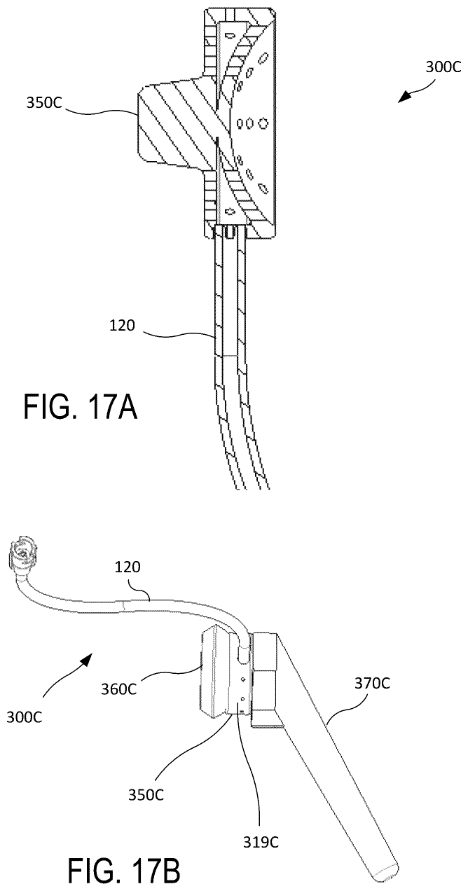

FIGS. 17A and 17B illustrate aspects of an exemplary reverse shoulder spacer system 300C. As depicted in the cross-section view of FIG. 17A, reverse shoulder spacer system 300C includes a reverse shoulder spacer 350C and a catheter 120. As shown in FIG. 17B, reverse shoulder spacer 350C includes a first surface configured for articulating engagement with a first implant (e.g. glenoid implant 360C), a second surface configured for fixed engagement with a second implant (e.g. humeral implant 370C), and an exposed surface 319C disposed between the first surface and the second surface. A reverse shoulder spacer 350C can include outlets through which treatment fluid may flow. For example, first surface can include outlets through which treatment fluid may flow toward a glenoid implant 360C, second surface can include outlets through which treatment fluid may flow toward a humeral implant 370C, and exposed surface 319C can include outlets through which treatment fluid may flow into a joint space between the glenoid implant 360C and the humeral implant 370C, for example to treat the nearby soft tissue.

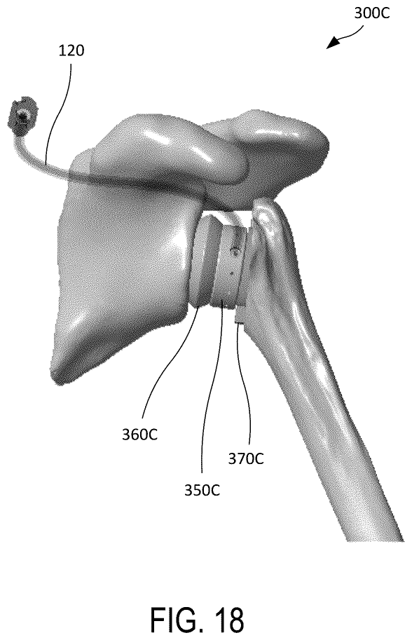

FIG. 18 depicts aspects of a reverse shoulder spacer system 300C, according to embodiments of the present invention. As shown here, the reverse shoulder spacer system 300C includes a reverse shoulder spacer 350C and a catheter 120. The reverse shoulder spacer 350C can engage a retained glenoid implant 360C which is attached with a glenoid bone. The reverse shoulder spacer 350C can also engage a retained humeral implant 370C which is attached with a humerus bone.

### Methods of Treating Periprosthetic Joint Infection

Provided herein are methods of treating periprosthetic joint infection, the method comprising: providing one of: the hip spacer platform, the knee spacer platform, and the shoulder spacer platform; and supplying a medication to the pump to irrigate a location of the joint infection over a period of time. In some embodiments, the medication is supplied continuously over a period of time. In some embodiments, the medication is supplied intermittently over a period of time. In some embodiments, the medication is removed by applying negative pressure continuously to the site of joint infection. In some embodiments, the medication is removed by applying negative pressure intermittently over a period of time.

In some embodiments, the hip spacer platform comprises a hip spacer, a catheter in fluidic communication with the hip spacer, and a pump in fluidic communication with the hip spacer and the catheter. In some embodiments, the knee spacer platform comprises a knee spacer, a catheter in fluidic communication with the knee spacer, and a pump in fluidic communication with the knee spacer and the catheter. In some embodiments, the shoulder spacer platform comprises a shoulder spacer, a catheter in fluidic communication with the shoulder spacer, and a pump in fluidic communication with the shoulder spacer and the catheter.

In some embodiments, the medication comprises a broad spectrum of antibiotics. In some embodiments, the medication comprises tobramycin sulfate, vancomycin HCl, or both. In some embodiments, the medication comprises tobramycin sulfate for a first period of time and vancomycin HCl for a second period of time. In some embodiments, the period of time is about 1, 2, 3, 4, 5, 6, 7, 8, 9, 10, 11, 12, 13, 14 or more days.

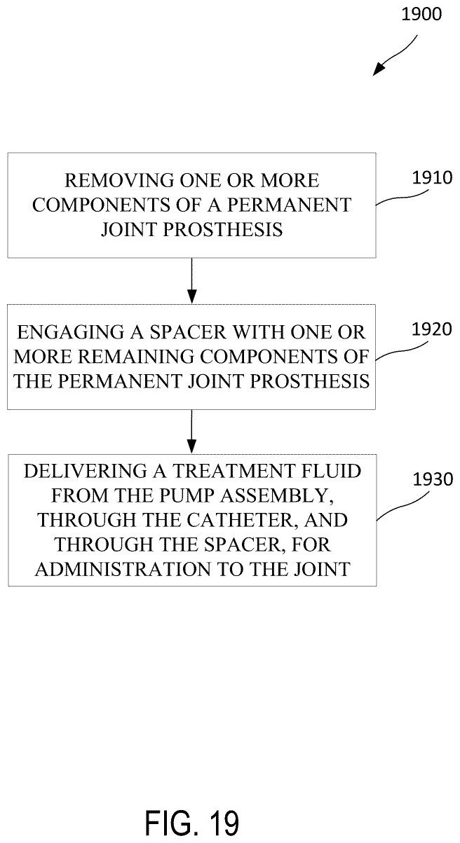

FIG. 19 depicts aspects of an exemplary method 1900 for treating a patient presenting with an acute periprosthetic joint infection of a joint, according to embodiments of the present invention. As shown here, the method 1900 can include removing one or more components of a permanent joint prosthesis, as depicted in step 1910. In some cases, a removal step may include removing one or more prosthetic components disposed between a first implant secured with a first bone of the joint and a second implant secured with a second bone of the joint. Methods may also include engaging a spacer of a spacer system with one or more remaining components of the permanent joint prosthesis, as depicted in step 1920. In some cases, the spacer system includes the spacer, a catheter, and a pump assembly. In some cases, the spacer and catheter are configured for detachable coupling. In some cases, the pump assembly is configured for coupling with the catheter. Methods may also include delivering a treatment fluid from the pump assembly, through the catheter, and through the spacer, for administration to the joint, as depicted in step 1930. In some cases, the delivery step 1930 can include delivering an antibiotic treatment fluid from the pump, through the catheter, into an inlet of the spacer. From the spacer, the fluid can flow out through a first plurality of outlets at a first surface of the spacer to the first implant, out through a second plurality of outlets at an exposed surface of the spacer and into a joint space between the first implant and the second implant, and out through a third plurality of outlets at a second surface of the spacer. The third plurality of outlets can be disposed within one or more fluted channels of the second surface so as to provide fluid flow between the spacer and the second implant. In some instances, the antibiotic treatment fluid is provided continuously or periodically to the patient over a treatment period of 7 days or more, and the catheter remains attached with the spacer throughout the treatment period

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Although embodiments of the present invention have been explained in relation to one or more preferred embodiments, it is to be understood that many other possible modifications and variations can be made without departing from the spirit and scope of the invention.

All features of the described systems and devices are applicable to the described methods mutatis mutandis, and vice versa.

Each reference provided herein in incorporated by reference in its entirety to the same extent as if each reference were individually incorporated by reference. Relatedly, all publications, patents, patent applications, journal articles, books, technical references, and the like mentioned in this specification are herein incorporated by reference to the same extent as if each individual publication, patent, patent application, journal article, book, technical reference, or the like was specifically and individually indicated to be incorporated by reference.

While the above provides a full and complete disclosure of exemplary embodiments of the present invention, which have been has been described in some detail by way of illustration and example for purposes of clarity of understanding, one of skill in the art will appreciate that certain changes, various modifications, alternate constructions, and/or equivalents may be practiced or employed as desired, and within the scope of the appended claims. Accordingly, the above description and illustrations should not be construed as limiting the invention, which can be defined by the claims.

1.-11. (canceled)

12. A method of treating a patient presenting with an acute periprosthetic joint infection of a joint, the method comprising:
removing one or more prosthetic components disposed between a first permanent implant secured within a first bone of the joint and a second permanent implant secured within a second bone of the joint;
placing a spacer of a spacer system between the first permanent implant and the second permanent implant, the spacer system comprising:
the spacer; and
a catheter,
wherein the spacer and the catheter are configured for detachable coupling; and
delivering a treatment fluid through the catheter, into an inlet of the spacer, and out through a first plurality of outlets at a first surface of the spacer to the first permanent implant, and out through a second plurality of outlets at an exposed surface of the spacer and into a joint space between the first permanent implant and the second permanent implant.

13. The method of claim 12, wherein the spacer system is a hip spacer system, the joint is a hip joint, the first permanent implant is an acetabular cup, and the second permanent implant is a femoral stem configured to couple with a femoral head.

14. The method of claim 12, wherein the spacer system is a knee spacer system, the joint is a knee joint, the first permanent implant is a femoral implant, and the second permanent implant is a tibial implant.

15. The method of claim 12, wherein the spacer system is a shoulder spacer system, the joint is a shoulder joint, the first permanent implant is a glenoid implant, and the second permanent implant is a humeral stem.

16. The method of claim 12, wherein the spacer further comprises a second surface, wherein the exposed surface is in a position between the first surface and the second surface.

17. The method of claim 16, further comprising engaging a convex shape of the first surface with a corresponding concave shape of the first permanent implant.

18. The method of claim 16, further comprising engaging a concave shape of the second surface with a corresponding convex shape of the second permanent implant.

19. The method of claim 16, wherein the first surface is configured for articulating engagement with the first permanent implant and wherein the second surface of the spacer is configured for articulating or fixed engagement with the second permanent implant.

20. The method of claim 16, wherein the second surface of the spacer comprises a plurality of fluted channels.

21. The method of claim 20, wherein at least one of the plurality of fluted channels comprises a plurality of fluid outlets.

22. The method of claim 20, wherein the plurality of fluted channels are arrayed about a center axis of the spacer.

23. The method of claim 20, further comprising enabling fluid flow under the spacer between the spacer and the second permanent implant via the plurality of fluted channels.

24. The method of claim 12, wherein the spacer is undersized relative to the first permanent implant.

25. The method of claim 12, wherein the spacer comprises a biocompatible polymer selected from the group consisting of low-density polyethylene (LDPE), high-density polyethylene (HDPE), polypropylene (PP), polyvinyl chloride (PVC) polystyrene (PS) nylon, polytetrafluoroethylene, a thermoplastic polyurethane (TPU), acetyl copolymer, silicone, polyether ether ketone (PEEK), a polyurethane, a biocompatible elastomer, and an ultrahigh molecular weight polyethylene (UHMWPE).

26. The method of claim 12, wherein delivering the treatment fluid comprises coupling to the catheter an infusion assembly to deliver the treatment fluid through the catheter.

27. The method of claim 26, wherein the infusion assembly comprises a gravity infusion and/or a pump assembly.

28. The method of claim 12, wherein the spacer further comprises a plurality of internal channels extending with the spacer, the plurality of internal channels configured in an array about a center axis of the spacer.

29. The method of claim 28, wherein the inlet is coaxial with a first internal channel of the plurality of internal channels.

30. The method of claim 29, wherein delivering the treatment fluid through the catheter comprises delivery of the treatment fluid into the first internal channel of the plurality of internal channels toward the center axis before exiting out through the second plurality of outlets into the joint space between the first permanent implant and the second permanent implant.

31. The method of claim 28, wherein each internal channel of the plurality of internal channels extends within the spacer and terminates at a respective outlet of either the first plurality of outlets through the first surface or the second plurality of outlets in the exposed surface.

32. The method of claim 12, wherein the inlet is through the exposed surface and the method further comprising detachably coupling the inlet to the catheter to receive the treatment fluid.

33. The method of claim 12, further comprising removing the spacer from between the first permanent implant and the second permanent implant after completion of delivering the treatment fluid through the catheter.

34. A method of treating a patient presenting with an acute periprosthetic joint infection of a joint, the method comprising:
removing one or more prosthetic components disposed between a first permanent implant secured within a first bone of the joint and a second permanent implant secured within a second bone of the joint;
placing a spacer of a spacer system between the first permanent implant and the second permanent implant, the spacer system comprising:
the spacer; and
a catheter,
wherein the spacer and the catheter are configured for detachable coupling, and wherein the catheter is configured for coupling with an infusion assembly; and
delivering a treatment fluid through the catheter, into an inlet of the spacer, and out through a first plurality of outlets at a first surface of the spacer to the first permanent implant, out through a second plurality of outlets at an exposed surface of the spacer and into a joint space between the first permanent implant and the second permanent implant, and out through a third plurality of outlets at a second surface of the spacer so as to provide fluid flow between the spacer and the second permanent implant,
wherein the treatment fluid is provided continuously or periodically to the patient over a treatment period of multiple days, and wherein the catheter remains attached with the spacer throughout the treatment period.

35. The method of claim 34, wherein the infusion assembly comprises gravity infusion and/or a pump assembly.

36. The method of claim 34, wherein the treatment period of multiple days comprises at least 7 days.

37. The method of claim 34, wherein the treatment fluid comprises antibiotic treatment fluid.

38. The method of claim 37, wherein the antibiotic treatment fluid comprises tobramycin, vancomycin, or both.

39. The method of claim 38, wherein the antibiotic treatment fluid comprises tobramycin for a first period followed by vancomycin for a second period.

40. The method of claim 34, wherein the spacer system is a hip spacer system, the joint is a hip joint, the first permanent implant is an acetabular cup, and the second permanent implant is a femoral stem configured to couple with a femoral head, or wherein the spacer system is a knee spacer system, the joint is a knee joint, the first permanent implant is a femoral implant, and the second permanent implant is a tibial implant, or wherein the spacer system is a shoulder spacer system, the joint is a shoulder joint, the first permanent implant is a glenoid implant, and the second permanent implant is a humeral stem.
